(19) United States
(12) Patent Application Publication
(10) Pub. No.: US 2011/0023586 A1
(43) Pub. Date: Feb. 3, 2011

(54) WATER ANALYSIS SENSOR ARRANGEMENT

(75) Inventors: Axel Leyer, Moenchengladbach (DE); Lothar Heidemanns, Korschenbroich (DE); Andreas Jonak, Meerbusch (DE); Markus Hahn, Kempen (DE); Michael Kussmann, Duesseldorf (DE); Heinz Rudde, Hueckelhoven (DE); Claudia Rieger, Duesseldorf (DE)

Correspondence Address: PATENT LAW OFFICES OF DR. NORMAN B. THOT, POSTFACH 10 17 56, RATINGEN, 40837, DE

(73) Assignee: Hach Lange GmbH, Berlin (DE)

(21) Appl. No.: 12/844,014

(22) Filed: Jul. 27, 2010

(30) Foreign Application Priority Data
Jul. 31, 2009 (EP) 09166939.0

(51) Int. Cl. G01N 33/18

(52) U.S. Cl. 73/53.01

(57) ABSTRACT

An immersion probe for water analysis includes at least two sensors configured to determine different water parameters, a sensor module receiving portion with at least two sensor-module plug-in positions, and at least two sensor modules. The at least two sensor-module plug-in positions are configured to be substantially physically and electrically similar. Each of the at least two sensor modules respectively comprise a sensor and are disposed in a respective sensor-module plug-in position.

### CROSS REFERENCE TO PRIOR APPLICATIONS

Priority is claimed to European Patent Application No. EP 09166939.0, filed Jul. 31, 2009. The entire disclosure of said application is incorporated by reference herein.

### FIELD

The present invention provides an immersion probe for water analysis comprising at least two different sensors for determining different water parameters.

### BACKGROUND

Immersion probes for water analysis are normally used for process control in water clarification and treatment or for continuous quality control of water. For this purpose, the water analysis immersion probe comprises different sensors which serve to determine the parameters of interest or are required for compensation of a main parameter. Depending on the respective application, different parameters or combinations of parameters of the water must always be determined. Thus, for each combination of parameters, a different water analysis immersion probe must be provided.

Such a water analysis immersion probe is described, for example, in EP 1 710 567 A1. This immersion probe comprises a sensor cartridge including three sensors for determining three different parameters.

For each differing combination of various sensors, a specially designed sensor cartridge must be constructed.

### SUMMARY

An aspect of the present invention is to provide an immersion probe for water analysis which is capable, with only minimum expenditure, to make different combinations of sensors available for use.

In an embodiment, the present invention provides an immersion probe for water analysis which includes at least two sensors configured to determine different water parameters, a sensor module receiving portion with at least two sensor-module plug-in positions, and at least two sensor modules. The at least two sensor-module plug-in positions are configured to be substantially physically and electrically similar. Each of the at least two sensor modules respectively comprise a sensor and are disposed in a respective sensor-module plug-in position.

### BRIEF DESCRIPTION OF THE DRAWINGS

The present invention is described in greater detail below on the basis of embodiments and of the drawings in which:

FIG. 1 is a view of a water analysis immersion probe comprising a sensor module receiving portion and four sensor modules adapted to be plugged in; and

FIG. 2 is a view of the water analysis immersion probe of FIG. 1 inclusive of plugged-in sensor modules in a schematic representation.

### DETAILED DESCRIPTION

The water analysis immersion probe of the present invention comprises a sensor module receiving portion with at least two sensor-module plug-in positions which are similar or identical with each other physically and electrically, for example, mechanically and with respect to their electric interface. The sensor module receiving portion can also comprise more than two, for example, three or four, identical sensor-module plug-in positions. There are also provided at least two sensor modules having the same physical design, for example, being largely identical in their external configuration, each of which are provided with a different sensor, wherein the sensor modules have been inserted into the said plug-in positions or are adapted for insertion thereinto. The sensor modules comprising different sensors also each have identical electric interfaces for electric connection to the sensor module receiving portion and, respectively, to the electric interface of the associated sensor-module plug-in position. The sensor modules with their different sensors are identical to each other only to the extent required for compatibility. The distal ends of the sensor module, however, do not always need to be of an identical design.

In the context of the present invention, the term “physically and electrically similar” is to be understood as denoting a similarity which safeguards that each sensor module will fit onto each sensor-module plug-in position, but not in the sense that the sensor modules must be of an identical design with regard to their respective sensor and the chemistry and electronics behind the sensor.

The sensor-module plug-in positions are adapted for insertion of any desired combination of sensor modules with different sensors. In this manner, the desired combination of sensors for a water analysis immersion probe can be conveniently combined for each intended application. The desired combination of sensors can be compiled or plugged together both by the manufacturer and by the customer.

The sensor module can, for example, comprise a redox sensor, a chloride sensor, a nitrate sensor, a potassium sensor, an enzyme sensor, an ammonium sensor or a pH sensor, or a corresponding membrane or another ion-selective membrane.

The sensor module receiving portion can, for example, comprise a temperature sensor. Alternatively or additionally, the sensor module receiving portion can comprise a reference electrode and an electrolyte bridge, for example, in the form of a so-called salt bridge, and a mass electrode. If the sensor module receiving portion comprises a reference electrode and an electrolyte bridge, the housing of the sensor module receiving portion can also serve as an electrolyte tank for the reference electrode and the electrolyte bridge.

On the sensor module receiving portion, there can, for example, be provided all the sensors and means which in connection with most of the various sensors are generally always needed. In this manner, the number of the plug-in positions and the technical expenditure required for them, particularly the required constructional space and the required means for the transmission of energy and information, can be restricted to the absolutely required minimum.

Each sensor module comprises an electronic sensor information memory containing information on the respective sensor. In the sensor information memory, information on the type of sensor as well as individual information on the sensor, such as calibration data, can be stored. Each sensor module further comprises a transmission means for transmission of said sensor information from the sensor information memory to the sensor module receiving portion. The transmission means can be realized as a wireless transmitter and/or, alternatively or additionally, by electric contacts co-operating with a corresponding transmission means on the sensor-module plug-in position on the side of the sensor module receiving portion. The transmission means of the sensor module receiving portion can be realized, for example, as a corresponding wireless transmitter/receiver component.

Each sensor module can further comprise an individual interior equipment as required for operation of the respective sensor and for evaluation of the sensor signal. The interior equipment can optionally comprise, for example, a tank containing an electrolyte or another liquid, a micropump, a measuring amplifier, evaluation electronics etc.

The sensor module can, for example, be formed as a disposable component. When exhausted or malfunctioning, the disposable sensor module can, for example, be removed and disposed of and be replaced by a corresponding new sensor module.

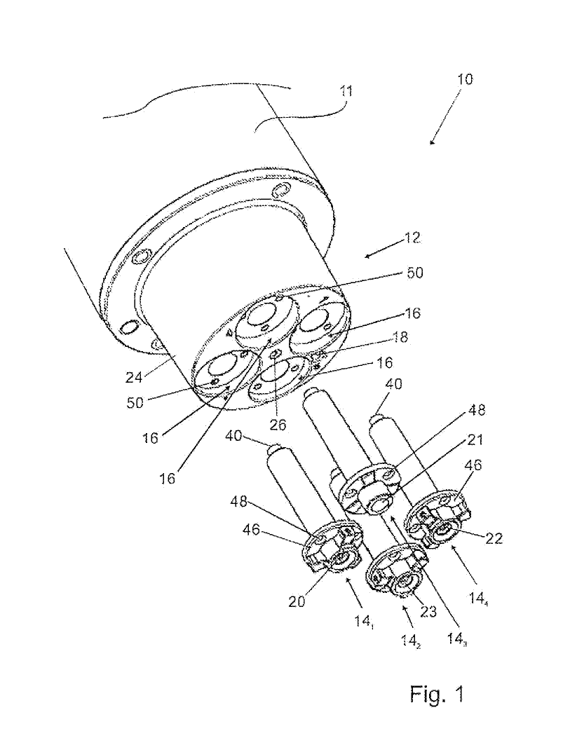

FIG. 1 is a perspective view of an immersion probe 10 for water analysis, provided for determination of four different water parameters. Said immersion probe 10 substantially consists of a probe base 11 and an exchangeable sensor module receiving portion 12 adapted for insertion of four individual sensor modules 14₁,14₂,14₃,14₄. For this purpose, the sensor module receiving portion 12 comprises four identical plug-in positions 16 into which said four sensor modules 14₁,14₂,14₃,14₄, being externally largely identical to each other, can be inserted or have been inserted.

With regard to their sensors 20,21,22,23 on the distal sensor module end, the four sensor modules 14₁,14₂,14₃,14₄ are different from each other. Said sensors 20,21,22,23 can be realized in the form of a redox electrode, a chloride electrode, a nitrate electrode, a potassium electrode, an enzyme electrode, an ammonium electrode, a pH electrode or another type of electrode, such as an ion-selective electrode. The electrodes of the sensors 20,21,22,23 can be formed, for example, as metal electrodes, solid-body membrane electrodes and/or PVC membrane electrodes.

In an embodiment of the present invention, the sensor modules 14₁,14₂,14₃,14₄ can, for example, be disposable components; i.e., they will be used only once and, when exhausted or defective, can be disposed of. Each sensor module 14₁,14₂,14₃,14₄ can be provided with a respective anti-twist protection device cooperating with a corresponding counterpart on plug-in position 16 for securement against rotation. Each sensor module 14₁,14₂,14₃,14₄ further comprises, at its distal end, an annular fastening flange 46 which in turn comprises three fastening bores 48. Attachment of a sensor module 14₁,14₂,14₃,14₄ is performed with the aid of corresponding threaded bolts which can be inserted into said fastening bores 48 and can be threaded into corresponding threaded bores 50 on the end side of the sensor module receiving portion 12. Between said fastening flange 46 and sensor base 12, a sealing ring can be provided for liquid-tight sealing of the annular space around sensor module 14₁,14₂,14₃,14₄ within plug-in position 16.

On the end side of the sensor module receiving portion 12, an electrolyte bridge can be provided, for example, in the form of a so-called salt bridge. The housing 24 of sensor module receiving portion 12 and a tank wall 31, arranged in a transverse plane and in the longitudinal direction located in the area of the plug-in position 16, together form an electrolyte tank 30 for an electrolyte which is in contact with the electrolyte bridge 18 and with a reference electrode immersed from above. Further, the sensor module receiving portion 12 comprises, on its end side, a temperature sensor 26 and can also comprise a mass electrode, not shown.

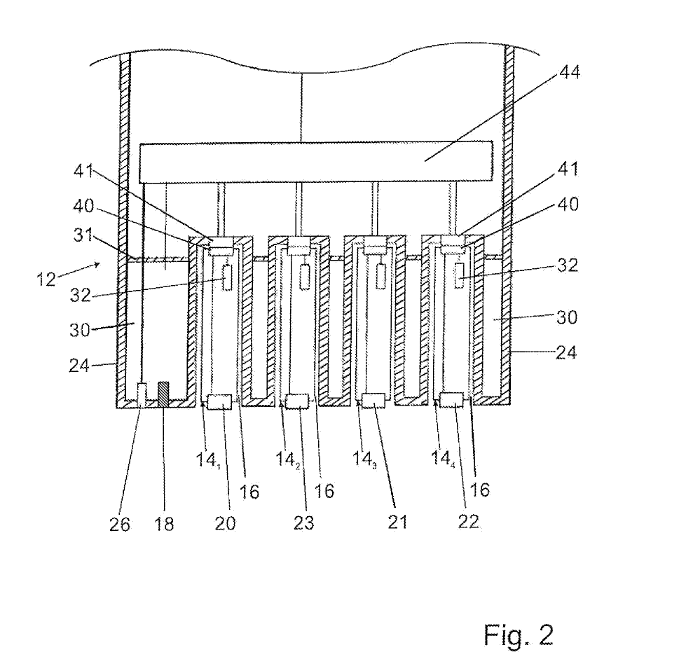

As illustrated in the schematic representation of FIG. 2, each sensor module 14₁,14₂,14₃,14₄ can comprise an electronic sensor information memory 32 in which information on the respective sensor 20,21,22,23, such as the type of sensor and calibration information, is stored.

For transmission of the sensor information which, apart from the information from said sensor information memory 32, also includes the measurement signal, each sensor module 14₁,14₂,14₃,14₄ can comprise an electric contact 40 as a transmission means which, after insertion of sensor module 14₁,14₂,14₃,14₄ into plug-in position 16, can be in contact with a corresponding electric terminal 41 of plug-in position 16. The electric terminals 41 of plug-in position 16, the temperature sensor 26 and the salt bridge 18 established via the electrolyte in electrolyte tank 30, can be electrically connected to a central control unit 44 provided to perform the control and surveillance of all components and the evaluation of the sensor signals.

As an alternative to the use of said electric terminals, the energy and the information can also be transmitted wirelessly via corresponding coils and via respective transmitter and receiver devices. In this case, the transmission means is a wireless transmitter.

The configuration of the water analysis immersion probe 10 for a special application or a special combination of water parameters to be determined can be realized by corresponding selection and placement of the sensor modules 14₁,14₂,14₃,14₄ on the sensor module receiving portion 12. The control unit 44 can read the information on the type of the respective sensor 20,21,22,23 from the respective sensor information memories 32; thus, mix-up or an incorrect manual input can be virtually excluded.

Although the present invention has been described and illustrated with reference to specific illustrative embodiments thereof, it is not intended that the present invention be limited to those illustrative embodiments. Those skilled in the art will recognize that variations and modifications can be made without departing from the true scope of the present invention as defined by the claims that follow. It is therefore intended to include within the present invention all such variations and modifications as fall within the scope of the appended claims and equivalents thereof.

1-12. (canceled)

13. An immersion probe for water analysis comprising:
at least two sensors configured to determine different water parameters;
a sensor module receiving portion with at least two sensor-module plug-in positions, the at least two sensor-module plug-in positions being configured to be substantially physically and electrically similar; and
at least two sensor modules each of which respectively comprises a sensor and each of which are disposed in a respective sensor-module plug-in position.

14. The immersion probe as recited in claim 13, wherein the sensor module receiving portion includes an electrolyte bridge.

15. The immersion probe as recited in claim 13, wherein the sensor module receiving portion includes an electrolyte tank filled with an electrolyte, the electrolyte tank being configured so as to be in contact with the electrolyte bridge.

16. The immersion probe as recited in claim 15, wherein the sensor module receiving portion includes a housing configured as the electrolyte tank.

17. The immersion probe as recited in claim 13, further comprising a probe base, the sensor module receiving portion being configured to be exchangeably fastened to the probe base.

18. The immersion probe as recited in claim 13, wherein the sensor module receiving portion includes a temperature sensor.

19. The immersion probe as recited in claim 13, wherein the sensor module receiving portion includes a mass electrode.

20. The immersion probe as recited in claim 13, wherein each of the at least two sensor modules includes an electronic sensor information memory configured to contain information on the respective sensor and a transmission device configured to transmit the information on the respective sensor to the sensor module receiving portion.

21. The immersion probe as recited in claim 20, wherein the transmission device is a wireless transmitter.

22. The immersion probe as recited in claim 20, wherein the transmission device comprises a first electric terminal on the respective sensor-module plug-in position and a second electric terminal on a sensor module.

23. The immersion probe as recited in claim 13, wherein the at least two sensor modules include at least one of a redox sensor, a chloride sensor, a nitrate sensor, a potassium sensor, an enzyme sensor, an ammonium sensor, a pH sensor and an ion-selective electrode.

24. The immersion probe as recited in claim 13, wherein the at least two sensor modules is a disposable component.
